(19) United States
(12) Patent Application Publication
(10) Pub. No.: US 2026/0151249 A1
(43) Pub. Date: Jun. 4, 2026

(54) PORTABLE ADJUSTABLE SUPPORT FOR AIDING IN THE REDUCTION OF ANTERIOR OR POSTERIOR SHOULDER DISLOCATIONS AND METHOD OF USING THE SAME

(71) Applicant: Product Innovations Inc., Aspen, CO (US)

(72) Inventors: Mark JOSEPH, Aspen, CO (US); Greogory Russel Balko, George, UT (US)

(21) Appl. No.: 19/406,574

(22) Filed: Dec. 2, 2025

Related U.S. Application Data
(60) Provisional application No. 63/726,762, filed on Dec. 2, 2024.

(51) Int. Cl. A61F 5/01; A45B 9/00; A45B 9/04; A63C 11/22

(52) U.S. Cl. CPC A61F 5/013; A45B 9/04; A63C 11/221; A45B 2009/007; A45B 2200/05

(57) ABSTRACT

A shoulder-reduction device and method of use are disclosed. The device is a portable, adjustable telescoping assembly configured to treat anterior or posterior shoulder dislocations by supporting the patient's body and shoulder during reduction. It includes an upper portion shaped to fit beneath the underarm, an upper telescoping tube, and a lower telescoping tube with a rigid support that rests on a chair or other stable surface. A flexible grip pad is attached to the rigid lower portion and is seated upon by the patient to stabilize the device. A cam-lock or similar mechanism engages a notched tube to fix the device at a selected height. When adjusted, the mid-support section holds the dislocated shoulder in a natural, supported position, allowing a practitioner to apply controlled pressure to relocate the shoulder quickly and with reduced effort. The device can be easily assembled, disassembled, and transported.

### CROSS REFERENCE TO RELATED APPLICATIONS

This application claims priority to Provisional Patent Application No. 63/726,762 filed on Dec. 2, 2024, the contents of which is incorporated herein by reference in its entirety.

### BACKGROUND OF THE INVENTION

### Field of the Invention

The present disclosure generally relates to an adjustable telescoping device that is used on a person with an anterior or posterior shoulder dislocation and method of using the same, so as to support the body and shoulder to aid in reduction and relocation of the joint by a qualified caregiver herein described as the shoulder reduction device.

### SUMMARY OF THE INVENTION

Accordingly, the invention is directed towards a portable Telescoping locking device fitted into the armpit that supports the body and shoulder during relocation of the joint.

The advantage of the invention is to provide a better way to reduce anterior or posterior shoulder dislocations than the typical methods involving multiple people, slings, bedsheets and the like in a more reproducible manner that is faster, and less painful for the patient.

Another advantage of the invention is that it can instantly be fitted to a variety of human sizes from children to large adults by means of an adjustable height telescoping or extending feature that can be locked to a specific height.

Another advantage of the invention is that it has an upper portion shaped to the underarm with a padded surface to comfortably support the body and shoulder.

Another advantage of the invention is that it has a bottom portion Intended to be set on a bed, chair, the ground, or other surfaces that the patient is seated on which includes a flexible high-grip pad portion that extends under the buttocks of the seated person to hold the device firmly in place.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, relate to the process of those trained in the process of anterior or posterior shoulder dislocation reduction (Provider/s), using the device to more dependably, more quickly, and with less patient pain, perform the reduction in varied environments. Providers may include trained physicians, physician's assistants, nurses, paramedics, athletic trainers, backcountry guides, or those qualified to provide the service. Anterior or posterior shoulder dislocations are source of frustration for both the Provider rendering care, and the patient. The historical way of treating them usually involves considerable time, cost, resources, and on occasion, the need for chemical sedation. All of these factors impose unnecessary discomfort and expense to the patient. The invention provides a solution that addresses all of these concerns. Patients with Anterior or posterior Shoulder dislocations are typically in a great deal of pain which has gone on for some time until they can be aided by a Provider, sometimes involving minutes or hours for transportation to a care facility. During this time, muscles often cease up trying to protect the injury and the further pressure causes further pain. Patients are typically stooped over towards the side the injury is on, holding their arm. The provider must get them seated and upright to have a chance of relocation. This typically requires two or more people to get the patient in the proper position for the Provider to hold their painful elbow and arm and manipulate the upper arm so as to cause the ball and socket joint to move into the proper position.

Training documents for medical professionals to be instructed in the process typically show up to ten various methods, all intended at the same results. These can be done sitting or lying down and multiple people may use various anatomical methods and positions using straps, bed sheets, and even a foot under the arm to achieve relocation. This takes considerable time to coordinate, and the patient can be in so much pain that chemical sedation may be required to reduce pain and resistance to the process. This often requires a blood test requiring further delay and it can take up to half an hour before the sedation takes effect. No products exist in the marketplace to aid in this agonizing process.

The invention and embodiment are directed towards quickly supporting the patient in a more upright evenly aligned position that pushes up into the armpit while being counter supported on the surface the patient is seated on. A telescoping or adjustable element of the device allows the Provider to instantly adjust the length of the device to properly support a wide variation of body sizes. The adjustment is intended to place the shoulders evenly horizontally aligned or for the injured shoulder to be slightly higher. Once in place, the patient typically feels the device provide support and often, instantaneous relief of pain, because the skeleton and muscles are relieved of supporting the injury. This can cause muscular and skeletal relaxation as the Provider instructs the patient to sit more upright, extend the chest forward and raise the head upright. Then, the provider can more gently and more quickly reduce the dislocation. This is a considerable difference from how most patients are typically positioned, often with multiple providers holding them up by hand.

Another aspect of the invention is that the process of properly supporting the patient, and performing the reduction, can often be done by a single individual. This is of particular advantage with large or unruly patients in pain who are slumped over. It is also an advantage for solo emergency Providers in the field which might include the backcountry, on snow, a playing field, in an ambulance or other environment. Once the proper position is achieved, the Provider manipulates the elbow and arm, typically providing downward pressure, with some rotational movement, to pull the shoulder back into place. The invention provides a fixed surface up under the armpit to apply this often-considerable pressure to. It is advantageous compared to using additional people to try to lift and apply counter body pressure for the Provider to work against. The process to adjust and install the device on the patient, and then perform the reduction, typically takes 1-5 minutes to perform and the support typically provides initial relief for the patient followed by an easier relocation.

And yet another aspect of the invention is that the device can be adapted to commonly carried devices used in the field such as a snow shovel, ski pole, walking stick, or other devices that would support the underarm pad.

And yet another aspect of the invention is that the device can be made from lightweight materials such as aluminum, injection molded plastic, composite reinforced plastics, various metals, wood, and even bamboo to be used in remote underprivileged areas of the world. The device can have multiple extendable sections that reduce the size, and advanced materials to make it very small and light weight for easy portage inside a backpack, first aid kit, rescue gear bag and the like.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1A is a perspective view of the shoulder reduction device, in accordance with an embodiment of the invention which illustrates the adjustable height set to the lowest position.

FIG. 1B is a perspective view of the shoulder reduction device, in accordance with an embodiment of the invention which illustrates the adjustable height set to the mid-height position.

FIG. 1C is a perspective view of the shoulder reduction device, in accordance with an embodiment of the invention which illustrates the adjustable height set to the highest position.

FIG. 1D is a cross-sectional view of the shoulder reduction device, in accordance with an embodiment, demonstrating the method and force direction a provider uses on a patient to reduce an anterior or posterior shoulder dislocation.

FIG. 2A is a side view of the shoulder reduction device, in accordance with an embodiment of the invention.

FIG. 2B is an alternate side view of the shoulder reduction device, in accordance with an embodiment of the invention.

FIG. 2C is top view of the shoulder reduction device, in accordance with an embodiment of the invention.

FIG. 2D is bottom view of the shoulder reduction device, in accordance with an embodiment of the invention.

FIG. 3A is a side view of the rigid underarm support, in accordance with an embodiment of the invention.

FIG. 3B is a bottom view of the rigid underarm support, in accordance with an embodiment of the invention.

FIG. 3C is an end view of the rigid underarm support, in accordance with an embodiment of the invention.

FIG. 3D is a perspective view of the flexible cushioned underarm pad that stretch fits over the rigid underarm support, in accordance with an embodiment of the invention.

FIG. 4A is a top perspective view of the base component of the shoulder reduction device, in accordance with an embodiment.

FIG. 4B is a bottom perspective view of the base component of the shoulder reduction device, in accordance with an embodiment.

FIG. 4C is a top view of the base component of the shoulder reduction device, in accordance with an embodiment.

FIG. 4D is a perspective view of the flexible pad component of the shoulder reduction device, in accordance with an embodiment.

FIG. 5 is an exploded view showing all of the components of the shoulder reduction device, in accordance with an embodiment of the invention.

FIG. 6 is a closeup view of the shoulder reduction device, in accordance with an embodiment of the invention showing the telescoping tubes with a U channel to keep them rotationally oriented.

FIG. 7A is a closeup view of the shoulder reduction device, in accordance with an embodiment of the invention showing a clamping lever locked to secure the shafts at a specific height.

FIG. 7B is a closeup view of the shoulder reduction device, in accordance with an embodiment of the invention showing a clamping lever unlocked to allow the shafts to slide to various heights.

FIG. 8A is a cross-sectional view of the shoulder reduction device, in accordance with an embodiment demonstrating the method to align and fit it to a patient with an anterior or posterior shoulder dislocation.

FIG. 8B is a cross-sectional view of the shoulder reduction device, in accordance with an embodiment, demonstrating the method to align the arm of a patient with an anterior or posterior shoulder dislocation.

FIG. 8C is a cross-sectional view of the shoulder reduction device, in accordance with an embodiment, demonstrating the method and force direction a provider uses on a patient to reduce an anterior or posterior shoulder dislocation.

FIG. 9 is a shoulder reduction device, in accordance with other embodiments of the invention incorporating one or more spikes to engage with outdoor surfaces.

FIG. 10A is a shoulder reduction device, in accordance with other embodiments of the invention incorporating three nesting tubes with two locking mechanisms

FIG. 10B is a shoulder reduction device, in accordance with other embodiments of the invention incorporating a nesting tube with a detent locking mechanism locking mechanism.

FIG. 10C is a shoulder reduction device, in accordance with other embodiments of the invention incorporating a scissor lift with a locking mechanism in the base.

FIG. 11A is a perspective view of the shoulder reduction device, in accordance with another embodiment that has a rigid metal base.

FIG. 11B is a perspective exploded view showing all of the components of the shoulder reduction device, in accordance with another embodiment that has a rigid metal base.

FIG. 11C is a perspective exploded view showing the three main components of the shoulder reduction device, that can be dissembled and reduced in size for carrying in accordance with another embodiment that has a rigid metal base.

FIG. 11D is a perspective rear view of the shoulder reduction device assembled in the form of a shovel, in accordance with another embodiment that has a rigid metal base.

FIG. 11E is a perspective front view of the shoulder reduction device assembled in the form of a shovel, in accordance with another embodiment that has a rigid metal base.

FIG. 12 is a perspective view of the shoulder reduction device, in accordance with another embodiment, that when assembled in an alternate configuration, uses a multi-part adjustable hiking or trekking pole as the telescoping support for the armpit pad.

FIG. 13 is a flowchart depicting a use of the shoulder reduction device in accordance with another embodiment.

### DETAILED DESCRIPTION OF THE ILLUSTRATED EMBODIMENTS

In the following description of embodiments, numerous specific details are set forth in order to provide more thorough understanding. However, note that the embodiments may be practiced without one or more of these specific details. In other instances, well-known features have not been described in detail to avoid unnecessarily complicating the description.

Embodiments are described herein with reference to the figures where like reference numbers indicate identical or functionally similar elements. Also, in the figures, the left most digits of each reference number correspond to the figures in which the reference number is first used.

One embodiment relates to a series of two or more telescoping nesting metal tubes that provide the adjustable height structure for the device. At the upper end of the first lowest tube, a locking device is affixed that can lock the second tube at any desired location. The preferred embodiment has two tubes that, when sized to the proper length, allow adjustment of the two tubes to fit from a child to a tall adult. An alternative embodiment has three or more nesting tubes to allow for the device to be made more compact for easier portage. The tubes can be nested in any order, small on top, or larger on top so long as they provide a telescoping feature.

In one embodiment, telescoping nesting tubes may have interlocking U indents running along the length to prevent the tubes from twisting so that the top and bottom components always stay properly aligned. Any configuration of tubes that are not completely round, and which prevent twisting between them can be used such as; square, oval, dog-bone and other nested shapes

In one embodiment, a series of holes in the outer shaft allow for a spring-loaded detent button located on the inner shaft to interlock with the various holes on the outer shaft. This allows for quick adjustment of the device to fit different sized people by depressing the button and sliding the shaft to another location awhile releasing the button to lock it into another hole.

In other embodiments, other means of adjusting the length can include hinged sections, scissor lift structures, snap together sections, twist lock telescoping tubes, and other means that achieve an adjustable height device. Additionally, the device could be non-adjustable in height and available in different sizes to fit a variety of human sizes. Whatever method of adjustment is used, it must be quick to adjust and withstand a load of at least 100 lbs (54.4 kg).

In one embodiment, the upper portion of the device includes an injection molded curved support that is permanently attached to the top of the adjustable height support and engages into the patient's armpit. It may be covered by a cushioning material such as rubber, soft polyurethane, polymer foam, fleece, or other material intended to provide comfort to the patient when downward pressure is applied. Some of these materials may be cored out and have thin ribs to flex and provide additional cushioning.

In one embodiment, the upper curved support may be detachable from the adjustable height support and quickly attached using a friction fit, détente button or another locking device. This is so it can be removed for a smaller packing footprint, or so the adjustable height support can be used for other purposes.

In one embodiment, the upper supporting, cushioned portion of the device is approximately 7 inch (17.8 cm) in length, 1½ (3.8 cm) in width, and 2 inch (5.1 cm) in depth. The depth dimension can include a curved shape that is approximately ½ inch (1.3 cm) lower at the center point. Of course, other dimensions larger and smaller can also be utilized. This curved shape is intended to accommodate the shape of the underarm much like a walking crutch might be shaped at the top. If the support is injection molded, it would be cored out with multiple supporting ribs for appropriate strength. The support could be made of other materials such as 3D printed plastic, hard polymer foam, metal, hard rubber, wood or other structural materials. The dimensions are an example of the preferred embodiment; however, other dimensions can be used so long as the surface provides enough support to comfortably load the arm to relocate the shoulder.

In one embodiment, the lower portion that contacts the surface that the patient is seated upon is a rigid support base that is bar shaped to provide a surface to absorb the load of the relocation process. It is also intended to provide stability to the device to make it easy to align and adjust to the patient. The support base is generally aligned parallel to the upper support. In many cases, the device will be used on a soft padded chair or hospital bed and placed touching or nearly touching the patient's hip so it needs to be large enough to spread the load so that the device does not move too much when the relocation loads are applied. In a preferred embodiment, the size of the bar is approximately 5 inch (12.7 cm) long, 1¼ inch (3.18 cm) wide and 1¼ inch (3.18 cm) tall, however other shapes and sizes can be used so long as they provide a similar function. If the support is injection molded, it would be cored out with multiple supporting ribs for appropriate strength. The support base could be made of other materials such as 3D printed plastic, hard polymer foam, metal, hard rubberwood or other structural materials.

In one embodiment, the support base has means to attach a soft flexible high-grip pad to the underside. The pad is placed under the buttock of the patient and is intended to stabilize the support base and to hold it in place as the device is adjusted in height and as the relocation process takes place. The pad is attached to the underside of the support base using rivets, snap fasteners, adhesive or other durable fastening means. In the preferred embodiment, the pad is approximately 5 inch (12.7 cm) wide by 7 inch (17.8 cm) long and ⅜ inch (4.8 mm). Of course, other dimensions larger and smaller can also be utilized. The size can vary so long as it performs the function to hold the device in place. The pad needs to be rigid enough to place easily under the patient, yet flexible enough to conform comfortably and grip to the buttock seated on a soft surface such as a chair, bed, grass, turf or other surface. It can also provide cushioning and comfort on a hard surface such as pavement or dirt. In the preferred embodiment, the pad is made of fiber reinforced non-toxic & 6p free PVC foam that is fiber reinforced, has high grip, is durable, and is often used in yoga mats. Other materials such as soft rubber or polymer, fiber reinforced soft rubber or polymer, polymer foam, thick fabric, silicone, or other durable material with minimal stretch.

In one embodiment, the support base does not have a soft high-grip flexible pad attached. It has pointed protrusions that are intended to provide grip and stability on surfaces such as grass, turf, dirt, wood benches, or other outdoor surfaces.

In one embodiment, the lower support base (without a soft pad) is a rigid wide rigid polymer plastic or metal that extends under the patient's buttock. This base can be in the shape of a rectangle or trapezoid and can have reinforcement elements such as turned up edges, ribs or other structural corrugations. The base can also be in the shape the spade of a shovel, snow shovel, or other shape.

Many other configurations of the device can be employed so long as there is an upper support that fits the armpit, an adjustable height stanchion to fit a variety of body sizes, and a lower base to support the loads that will be applied during shoulder relocation.

Persons skilled in the art will readily appreciate that various aspects of the present disclosure can be realized by any number of methods and articles configured to perform the intended functions. Stated differently, other methods and articles can be incorporated herein to perform the intended functions. It should also be noted that the accompanying drawing figures referred to herein are not all drawn to scale but may be exaggerated to illustrate various aspects of the present disclosure, and in that regard, the drawings should not be construed as limiting. Finally, although the present disclosure may be described in connection with various principles and beliefs, the present disclosure should not be bound by any theory.

Reducing anterior or posterior shoulder dislocations is typically done by a qualified provider using a number of techniques that can include several people, chairs, bedsheets and even a foot in the armpit. The process can be very painful and time consuming requiring several attempts to hold the patient in position and apply enough force to perform the task. A better solution is needed. There are no devices currently available that are intended to aid in the reduction and relocation of such injuries. The exemplary embodiments of the present disclosure is a device to quickly and reliably perform shoulder reductions that answers this need.

Reference will now be made in detail to an embodiment of the present invention, example of which is illustrated in the accompanying drawings.

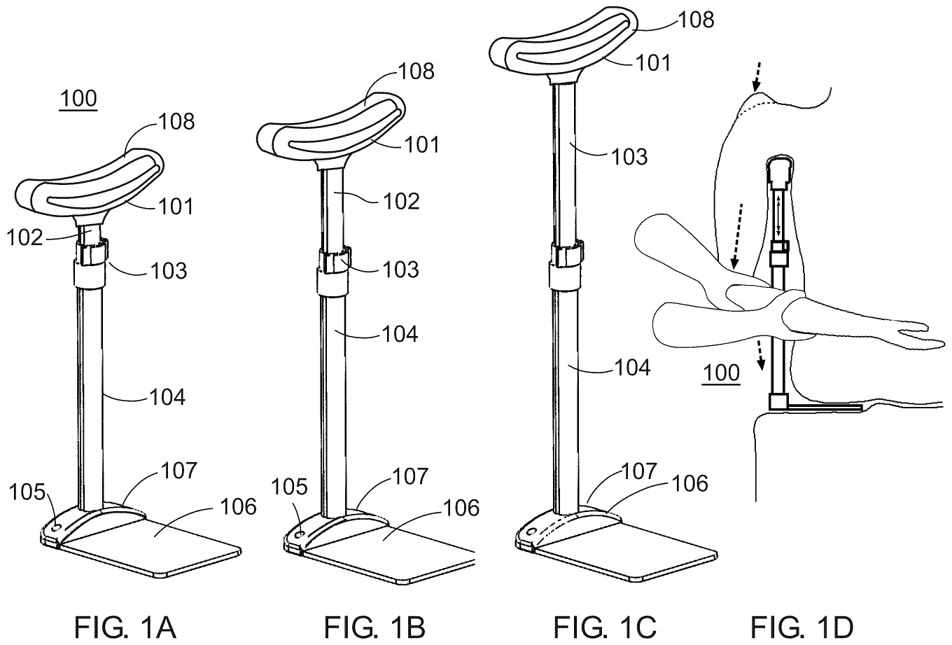

FIG. 1A is a perspective view of the shoulder reduction device, in accordance with an embodiment of the invention which illustrates the adjustable height set to the lowest position. FIG. 1B is a perspective view of the shoulder reduction device, in accordance with an embodiment of the invention which illustrates the adjustable height set to the mid-height position. FIG. 1C is a perspective view of the shoulder reduction device, in accordance with an embodiment of the invention which illustrates the adjustable height set to the highest position. FIG. 1D is a cross-sectional view of the shoulder reduction device, in accordance with an embodiment, demonstrating the method and force direction a provider uses on a patient to reduce an anterior or posterior shoulder dislocation.

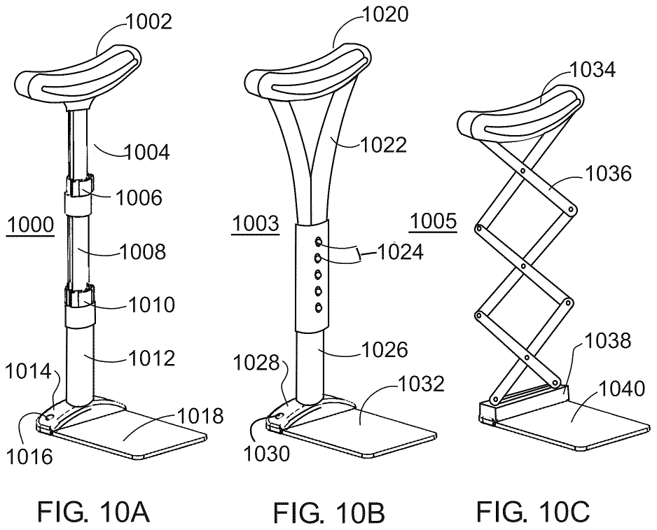

Referring to FIGS. 1A-1D, a shoulder reduction device is generally shown with reference to number 100 in various heights. The shoulder reduction device is adjustable vertically to accommodate a wide variety of patients. FIG. 1A shows the device FIG. in the lowest position. The shoulder reduction device or device 100 includes an underarm support 101, an upper telescoping tube 102, a lower, telescoping tube 104, a base 107, and an optional flexible pad 106. The upper telescoping tube 102 is configured to be inserted into lower tube 104. A cam lock lever fitting 103 or other types of locking mechanisms, such as a detent button, or twisting threaded compression lock, is securely clamped to lower tube 104. Cam lock lever 103 can be opened and closed to allow the tubes to slide within each other to a desired height while closing the lever secures the tubes to each other supporting at least 120 lbs. (54.3 kg). In one embodiment, the lower tube 104 is configured to be inserted into a rigid base 107 is attached by an interference tight fit, adhesive, rivets, screws or other securing means. Flexible pad 106 is secured to base 107 by rivets 105 which can be in the form of metal pop rivets, plastic mushroom press fit rivets, screws or the like. Upper tube 102 is configured to be inserted into underarm support with padded surface 101 and is attached by means of an interference tight fit, adhesive, rivets, screws or other securing means. FIG. 1B depicts the telescoping feature extended to a midway height. FIG. 1C depicts the telescoping feature extended to the maximum height. FIG. 1D depicts the embodiment of the device being installed and used on a patient showing the forces to be applied to be further described with reference to FIG. 10.

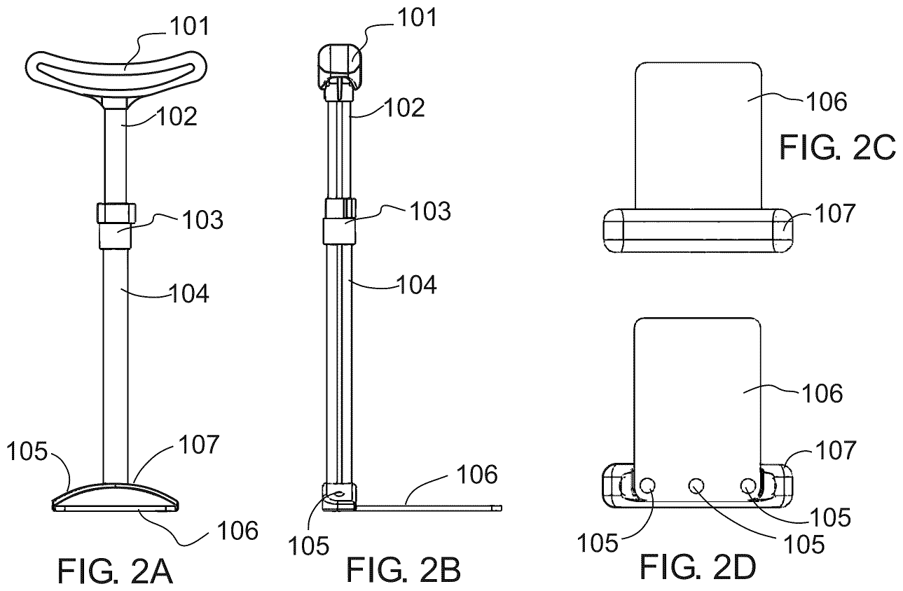

FIG. 2A is a side view of the shoulder reduction device, in accordance with an embodiment of the invention. FIG. 2B is an alternate side view of the shoulder reduction device, in accordance with an embodiment of the invention. FIG. 2C is top view of the shoulder reduction device, in accordance with an embodiment of the invention. FIG. 2D is bottom view of the shoulder reduction device, in accordance with an embodiment of the invention.

Referring to FIGS. 2A-2D showing different view of FIGS. 1A-1D and illustrating the underarm support 101, an upper telescoping tube 102, a lower, telescoping tube 104, a base 107, and an optional flexible pad 106. Flexible pad 106 is secured to base 107 by rivets 105 which can be in the form of metal pop rivets, plastic mushroom press fit rivets, screws or the like. Again, the flexible pad 106 is secured to base 107 by rivets 105 which can be in the form of metal pop rivets, plastic mushroom press fit rivets, screws or the like. The number of rivets 105 in this embodiment is three, however, any number of rivets 105 may be utilized to secure the flexible pad 106 to the base 107. The flexible pad can be constructed from a compliant, resilient cushioning material such as foam, gel, silicone, neoprene, viscoelastic gel, closed-cell polyurethane foam, thermoplastic elastomer (TPE), medical-grade silicone elastomers, textile-backed elastomeric composites, and equivalents thereof. These materials are configured to conform to the user's anatomy, distribute load across the axilla, and reduce localized pressure during use. The flexible pad 106 may also be configured to be releasable with the base allowing a user to remove it from the base as desired.

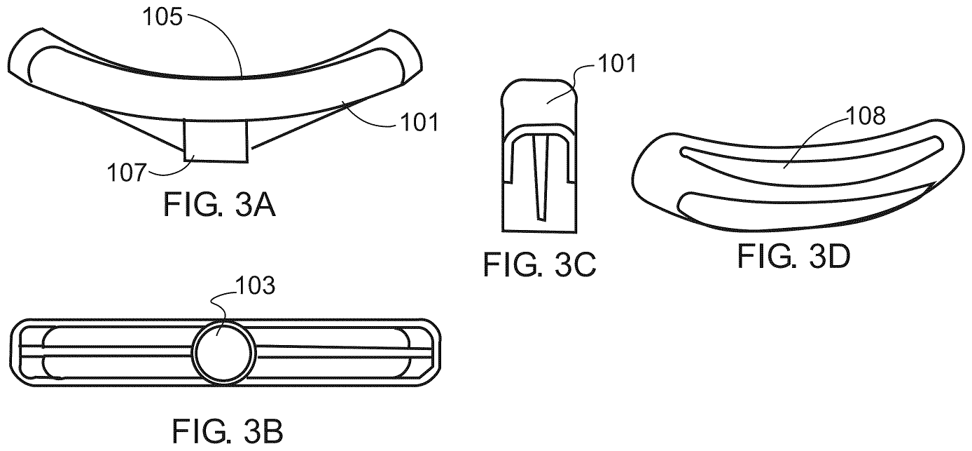

FIG. 3A is a side view of the rigid underarm support, in accordance with an embodiment of the invention. FIG. 3B is a bottom view of the rigid underarm support, in accordance with an embodiment of the invention. FIG. 3C is an end view of the rigid underarm support, in accordance with an embodiment of the invention. FIG. 3D is a perspective view of the flexible cushioned underarm pad that stretch fits over the rigid underarm support, in accordance with an embodiment of the invention.

Referring to FIGS. 3A-3D, the a rigid underarm support 101 is shown having top 105 and bottom 107. The bottom 107 has a hole 103 configured to receive a portion of the upper telescoping pole 102. The pole 102 may be secured with a locking mechanism, e.g., screw, rivet or the like.

The rigid underarm support 101 can be made of plastic including but not limited to nylon, polypropylene, and other durable polymers and can include carbon or glass fiber. It can be made by injection molding, 3D printing, compression molding or machining.

The rigid underarm support 101 optionally includes a flexible underarm cushion cover 108 that is configured to stretch fits over the rigid arm support 101. The flexible underarm cushion 108 can be made of soft materials that absorb shock such as natural rubber, polyurethane, synthetic rubber, EVA, and latex. These materials can be foam injected to add cushioning and reduce weight.

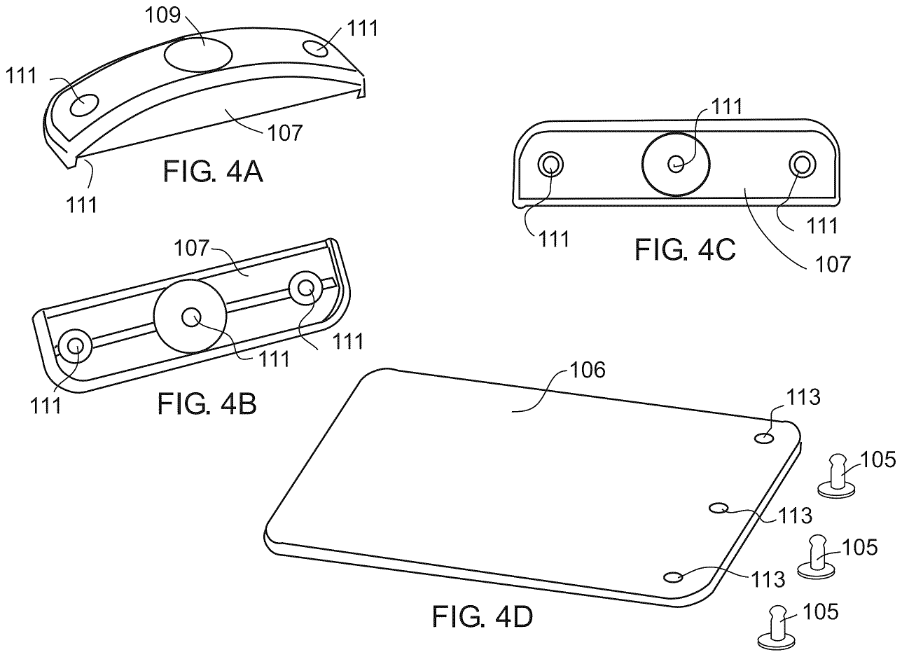

FIG. 4A is a top perspective view of the base component of the shoulder reduction device, in accordance with an embodiment. FIG. 4B is a bottom perspective view of the base component of the shoulder reduction device, in accordance with an embodiment. FIG. 4C is a top view of the base component of the shoulder reduction device, in accordance with an embodiment. FIG. 4D is a perspective view of the flexible pad component of the shoulder reduction device, in accordance with an embodiment.

Referring to FIGS. 4A-4D, is the base support 107 including holes 111 for the rivets 105 for connecting the pad 106 with holes 113 in the pad and base. In addition, the lower telescoping pole 104 is sized to fit in hole 109 and optionally a securing mechanism can be used to secure it.

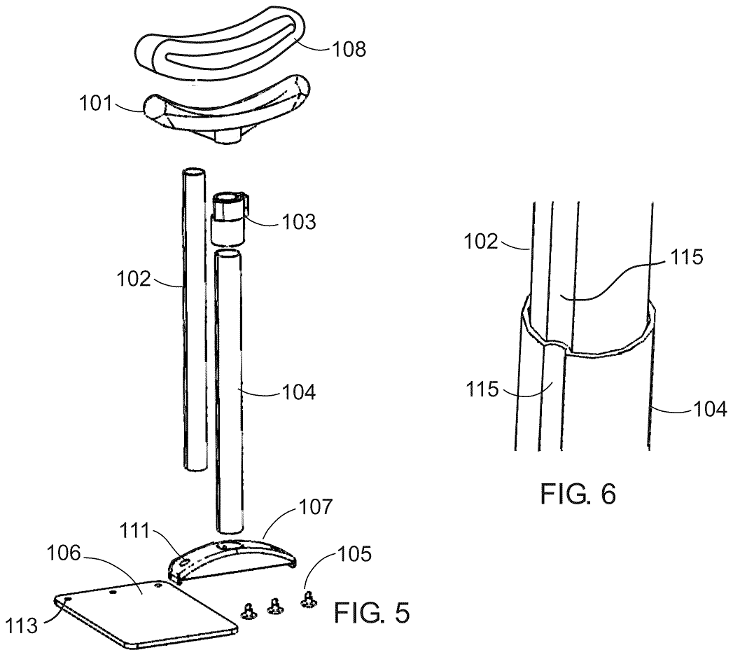

FIG. 5 is an exploded view showing all of the components of the shoulder reduction device, in accordance with an embodiment of the invention. FIG. 6 is a closeup view of the shoulder reduction device, in accordance with an embodiment of the invention showing the telescoping tubes with a U channel to keep them rotationally oriented;

Referring to FIGS. 5-6, the shoulder reduction device 100 is shown in a disassembled, portable configuration. The device 100 is designed to be easily transported, assembled, and adjusted, and includes an underarm support 101, an upper telescoping tube 102, a lower telescoping tube 104, a base 107, and an optional flexible pad 106. The upper telescoping tube 102 is configured to be inserted into the lower telescoping tube 104 to allow height adjustment and compact storage. A cam-lock lever fitting 103, or another suitable locking mechanism such as a detent button, or twisting threaded compression lock, is secured to the telescoping tubes to lock them in place during use and to permit quick release for disassembly. The notched 115 tubes 104 and 102 are configured to prevent rotational movement between the telescoping components during use.

Referring to FIGS. 5-6, the shoulder reduction device 100 is shown in a dissembled configuration. are side and end views of the base support 105. FIG. 4F is a perspective view of the flexible base pad. FIG. 5 depicts an embodiment of an embodiment of the device in an exploded view. FIG. 6 depicts an embodiment of the telescoping tubes with a pair of interlocking U channels to keep the upper and lower tubes aligned. Other shapes can be used, but not limited to squares, rectangles, ovals, dog bone shapes, and the like.

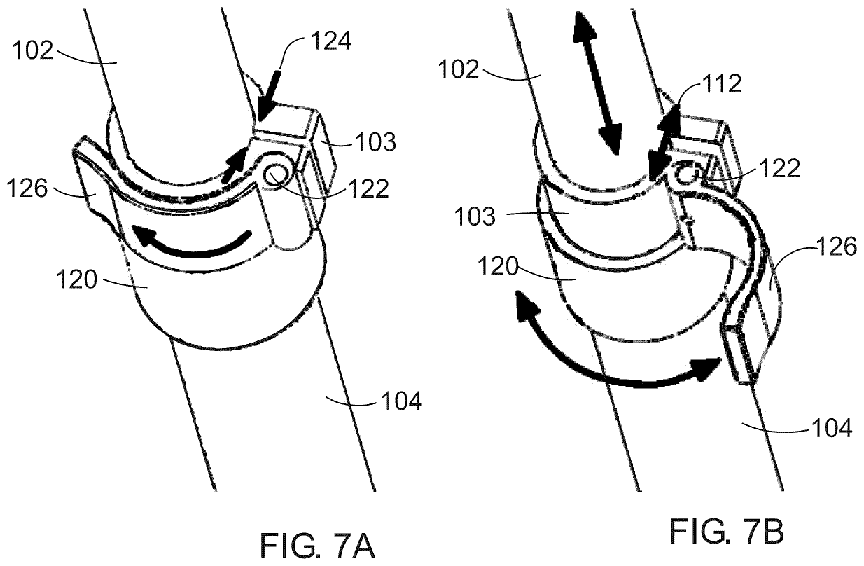

FIG. 7A is a closeup view of the shoulder reduction device, in accordance with an embodiment of the invention showing a clamping lever locked to secure the shafts at a specific height. FIG. 7B is a closeup view of the shoulder reduction device, in accordance with an embodiment of the invention showing a clamping lever unlocked to allow the shafts to slide to various heights.

Referring to FIGS. 7A-7B, the tube locking mechanism 103 includes a lower portion 120 that is clamped to the lower tube 104 with a compression bolt, rivet, adhesive or the like. Cam lever 126 in the closed position that is attached to an axis pin 122 that resides in the upper portion of 103 that is attached to an adjustable bolt, not shown, that provides clamping force to the upper portion as demonstrated by the arrows 124 when the lever is closed. FIG. 7B depicts the cam lever 126 open, thereby loosening the upper portion of clamp 103 as demonstrated by arrows 112, allowing upper tube 102 to slide freely within lower tube 104.

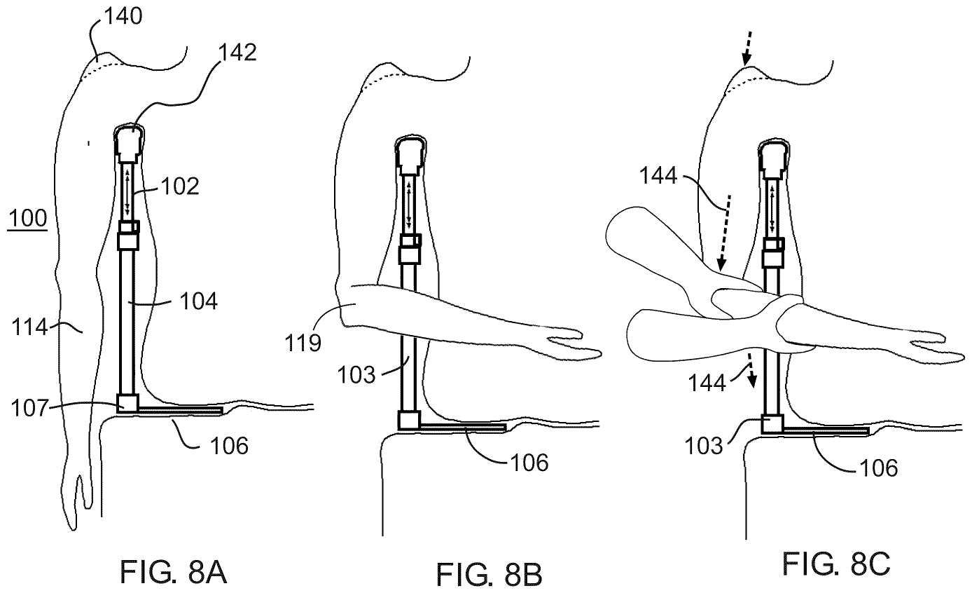

FIG. 8A is a cross-sectional view of the shoulder reduction device, in accordance with an embodiment demonstrating the method to align and fit it to a patient with an anterior or posterior shoulder dislocation. FIG. 8B is a cross-sectional view of the shoulder reduction device, in accordance with an embodiment, demonstrating the method to align the arm of a patient with an anterior or posterior shoulder dislocation. FIG. 8C is a cross-sectional view of the shoulder reduction device, in accordance with an embodiment, demonstrating the method and force direction a provider uses on a patient to reduce an anterior or posterior shoulder dislocation.

Referring FIGS. 8A-C, the shoulder reduction device 100 is arranged with a patient under an armpit of the patient with an anterior or posterior shoulder dislocation 140 so that the height of the telescoping tubes 102 an 104 and underarm support 101 with cushioned pad 106 lifts and supports the underarm 142 into a comfortable and anatomical position, so the patient can relax and not be using muscles which are in spasm, to support the shoulder. The base support 107 is arranged near the patient's hip while seated with the flexible pad 106 sandwiched between the patient's buttock and the seat so as to secure its position. The elbow 119 is bent into the typical position prior to the provider holding the arm for reduction of the dislocation. With the elbow 119 bent into the typical position with the provider holding the arm with two hands in a typical position for reduction of the dislocation. Pressure, downward load, and a twisting motion, as depicted by the dashed arrows 144, are typically used to cause the shoulder joint to reduce it into the proper position, thereby providing rapid relief for the patient.

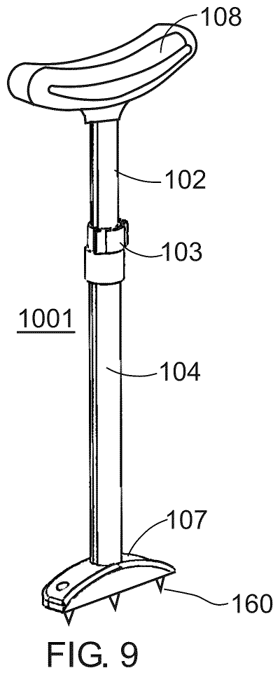

FIG. 9 is a shoulder reduction device, in accordance with other embodiments of the invention incorporating one or more spikes to engage with outdoor surfaces.

Referring to FIG. 9, the base 107 of the device 1001 incorporates one or more spikes 160 cable of engaging with rough outdoor surfaces such as grass, dirt, ice, snow and other natural surfaces without sliding from the patient. The spikes 160 may be sharp, and semi sharp or in cylindrical poles with grip materials.

FIG. 10A is a shoulder reduction device, in accordance with other embodiments of the invention incorporating three nesting tubes with two locking mechanisms.

Referring to FIG. 10A a shoulder reduction device is generally shown with reference to number 1000 in various heights. The shoulder reduction device 1000 is adjustable vertically to accommodate a wide variety of patients. The device 1000 includes an underarm support 1002, an upper telescoping tube 1004, a first locking mechanism 1006, e.g., a cam lock lever, a middle telescoping tube 1008, a second locking mechanism 1010, e.g., a cam lock lever fitting, a bottom tube 1012, a base 1014, and a flexible pad 1018. The upper telescoping tube 1004 is configured to be inserted into middle tube 1008. A cam lock lever fitting 1006 or type of locking mechanism such as a detent button, or twisting threaded compression lock, is securely clamped to the middle tube 1008. Cam lock lever 1006 can be opened and closed to allow the tubes to slide within each other to a desired height while closing the lever secures the tubes to each other supporting at least 120 lbs. (54.3 kg).

A middle tube 1008 is configured to be inserted into the base tube 1012. A cam lock lever fitting 1010 or type of locking mechanism such as a detent button, or twisting threaded compression lock, is securely clamped to the middle tube 1008. Cam lock lever 1008 can be opened and closed to allow the tubes to slide within each other to a desired height while closing the lever secures the tubes to each other supporting at least 120 lbs (54.3 kg)

The lower tube 1012 is configured to be inserted into a rigid base 1014 and attached by an interference tight fit, adhesive, rivets, screws or other securing mechanism. Flexible pad 1018 is secured to base 1014 by rivets 1016 which can be in the form of metal pop rivets, plastic mushroom press fit rivets, screws or the like. Upper tube 1004 is configured to be inserted into underarm support 1002 with padded surface and is attached by means of an interference tight fit, adhesive, rivets, screws or other securing means. The device is used on patient as described herein and as further described with reference to FIG. 10.

FIG. 10B is a shoulder reduction device, in accordance with other embodiments of the invention incorporating a nesting tube with a detent locking mechanism locking mechanism.

Referring to FIG. 10B, a shoulder reduction device is generally shown with reference to number 1003. The shoulder reduction device 1003 is adjustable vertically to accommodate a wide variety of patients. The device 1003 includes an underarm support 1020, an upper tube 1022 have y-shape, a first locking mechanism 1006, e.g., a nesting tube with a detent locking mechanism locking mechanism with a number of different locking positions 1024, a lower tube 1026, a base 1028, and a flexible pad 1032.

The upper tube 1022 is configured to be inserted over a portion of the lower tube 1026. The lower tube 1032 is configured to be inserted into a rigid base 1028 and attached by an interference tight fit, adhesive, rivets, screws or other securing mechanism. Flexible pad 1032 is secured to base 1028 by rivets 1030 which can be in the form of metal pop rivets, plastic mushroom press fit rivets, screws or the like. Upper tube 1022 is configured to be inserted into underarm support 1020 with padded surface and is attached by means of an interference tight fit, adhesive, rivets, screws or other securing means. The device is used on patient as described herein and as further described with reference to FIG. 10.

FIG. 10C is a shoulder reduction device, in accordance with other embodiments of the invention incorporating a scissor lift with a locking mechanism in the base.

Referring to FIG. 10C a shoulder reduction device is generally shown with reference to number 1005 in various heights. The shoulder reduction device 1005 is adjustable vertically to accommodate a wide variety of patients. The device 1005 includes an underarm support 1034, a scissor lift 1036 with a locking mechanism in the base 1038. The upper portion of the scissor lift 1036 is configured to be inserted into underarm support 1002 with padded surface and is attached by means of an interference rivets, screws or other securing means. The lower portion of the scissor lift 1036 is configured to be inserted into the base 1038 by means of an interference rivets, screws or other securing means. The base is also attached to a flexible pad 1040. The device is used on patient as described herein and as further described with reference to FIG. 10.

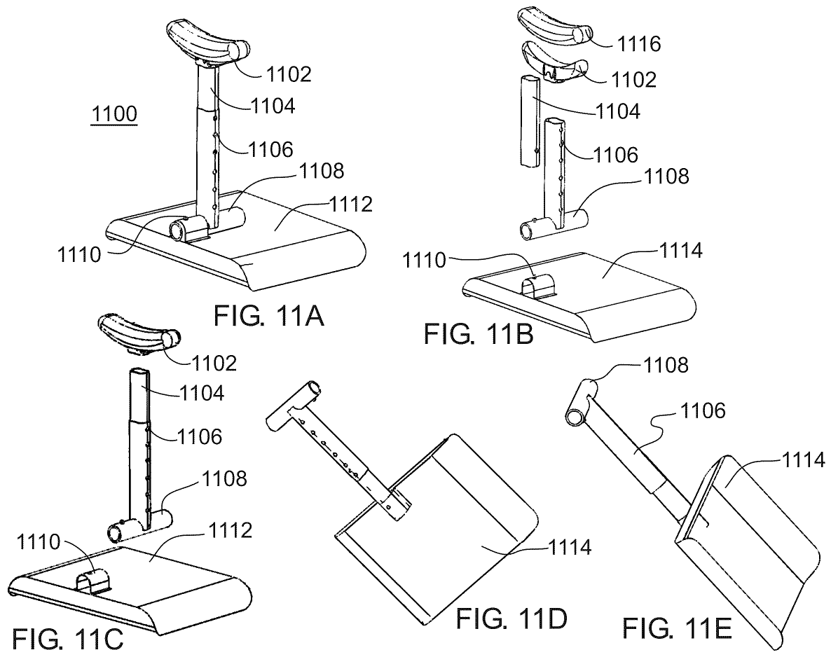

FIG. 11A is a perspective view of the shoulder reduction device, in accordance with another embodiment that has a rigid metal base. FIG. 11B is a perspective exploded view showing all of the components of the shoulder reduction device, in accordance with another embodiment that has a rigid metal base. FIG. 11C is a perspective exploded view showing the three main components of the shoulder reduction device, that can be dissembled and reduced in size for carrying in accordance with another embodiment that has a rigid metal base. another embodiment that has a rigid metal base.

Referring to FIGS. 11A-11E, a shoulder reduction device or device 1100 is a portable device configured to fit within a small carrying case, e.g., backpack. The device 1100 is also dual purpose and can be utilized as a shovel. The device 1100 includes an underarm support 1102, an upper telescoping tube 1104, a lower, telescoping tube 1106, a T-bar 1108, and a dual purpose base 1112. The upper telescoping tube 1104 is configured to be inserted into lower tube 1108. A locking mechanism, e.g., button lock, or other type locking mechanism such as a detent button, or twisting threaded compression lock, secures the lower tube 1106 to the upper tube 1104. The T-bar 1108 allows the lower tube to be removably secured to the base 1112. The base is shaped to have a cutout region on one side 114 to be used as a shovel as shown in FIGS. 11D-11E.

In one embodiment, the device 1100 is configuration allow the shoulder reduction device 1110 to be fitted to an item that is typically carried by guides, outdoor medics, ski patrollers and the like such as a shovel 1114 without having to carry the entire device separately. The shoulder support 1102 is the only extra item required thereby reducing bulk and weight. Other configurations can be done in a similar fashion using typical items carried by outdoor enthusiasts, athletic trainers, patrollers, medics, army medics, doctors and EMT technicians. The device being installed and used on a patient showing the forces to be applied to be further described with reference to FIG. 10. The device can arranged into a shovel and used a shovel, e.g., a snow shovel, as shown in FIGS. 11D-11E.

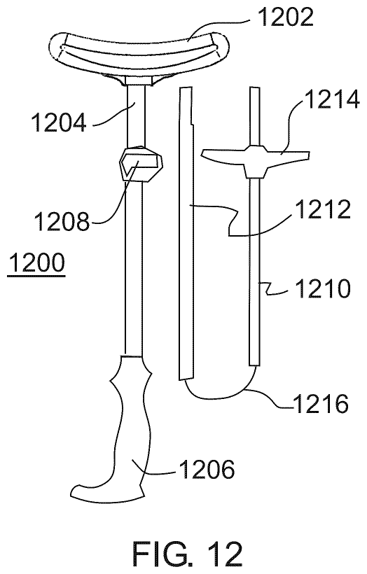

FIG. 12 is a perspective view of the shoulder reduction device, in accordance with another embodiment, that when assembled in an alternate configuration, uses a multi-part adjustable hiking or trekking pole as the telescoping support for the armpit pad.

Referring to FIG. 12, the portable device 1200 is configured to being temporarily affixed to a three-part trekking pole that is adjustable in height which can be used while sitting on dirt, rocks or other hard outdoor surfaces. The device 1200 includes an underarm support 1202, a tube 1204, a base and handle 1206. Of course a pad for the handle can be utilized. The device also includes two additional poles 1212 and 1210 a basket 1214. A wire 1216 connects the pole to keep them together in a spring like manner. The poles are for trekking and configured to attach to the handle 1206.

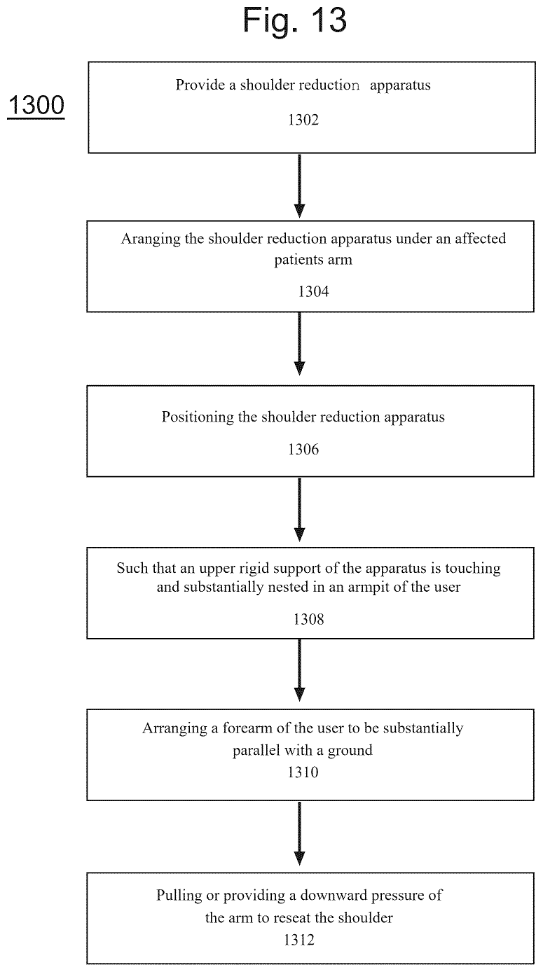

FIG. 13 is a flowchart depicting a use of the shoulder reduction device in accordance with another embodiment.

Referring to FIG. 13, the process 1300 is a method of shoulder reduction and includes provide a shoulder reduction apparatus (step 1302), arranging the shoulder reduction apparatus under an affected patients arm (step 1304), positioning the shoulder reduction apparatus (step 1306) such that an upper rigid support of the apparatus is touching and substantially nested in an armpit of the user (step 1308), arranging a forearm of the user to be substantially parallel with a ground (step 1310), pulling or providing a downward pressure of the arm to reseat the shoulder (step 1312). In a preferred embodiment, the user in a seated position during this shoulder relocation process.

By way of further description and in one embodiment, the use of the shoulder reduction device as commonly performed in a hospital emergency room, urgent care center or other indoor facility. The patient enters the care facility with a typical anterior or posterior shoulder dislocation that occurred on a downhill ski area due to a fall. He is in a great deal of pain and the shoulder has been dislocated for almost an hour during the ambulance ride and check-in. His right shoulder is dropped over as the muscles have contracted attempting to stabilize the injury which is partly what is causing the extreme pain. He is holding his arm and slouched forward and to the right.

The physician examines the patent to determine that it is a dislocation only, without a broken clavicle or other bones. He determines that the shoulder must quickly be reduced to realign the joint. He could call over two or more assistants to help hold the patient up and provide the proper counter leverage that is needed to perform the relocation. He could also order a sedative to reduce pain and relax the gripping muscles but this can take up to 45 minutes and also require a prior blood test.

The physician decides to use the shoulder reduction device to support the patient to perform the reduction because other personnel are occupied. He has the patient sit on a bench that does not have arm rests. He slides the flexible high-grip mat under the patient's right buttock as he sits down so that the lower support base is parallel to the femur bone and close to the hip. He loosens the locking collar attached to the lower outer telescoping tube so that the upper tube and armpit support can be adjusted to the proper height. U shaped indents in the telescoping tubes prevent rotation to keep the upper and lower sections aligned. In seconds, the physician adjusts the upper armpit pad snuggly under the patient's armpit and gently lifts it up so that the patient's shoulders are parallel with the ground and he is not slumped over to the right. He locks the collar clamping the tubes together and checks the height for proper alignment. It is often best to have the right shoulder just slightly higher than the left one since, when downward pressure is applied during the procedure, it may compress the cushioned surface the patient is seated on.

In most instances, sitting with the device, the patient receives immediate relief because they no longer need to support the arm and should themselves. The physician instructs the patient to sit as upright as possible with their head up straight and their chest extended upwards and outwards and then to breath as deep as possible. He rechecks the alignment and adjustment and then is ready to perform the reduction. There are many motions and applied loads that may cause the shoulder to relocate and the physician uses their experience to pick the proper one. The shoulder reduction device is capable of providing support and counter force to most of the techniques. In this case, the physician stands to the side of the patient and places his left hand around the elbow, or perhaps inside the crux of the elbow, and the right hand around the patient's forearm. He gently applies downward pressure which transfers up the arm, onto the curved armpit pad and down through the device to the seat. As this s done, the arm can be rotated by moving the forearm back and forth horizontally. Additionally, the entire arm might be rotated around towards the front of the body as downward pressure is applied. The physician “feels” the alignment, and with experience, quickly causes the joint to slip back into place, often with a popping sound. The patient immediately feels pain relief and relaxation along with more arm sensation. They can sit with the device in place, take a few breaths and have their shoulder mobility back to a degree as the muscles loosen back to a more normal state.

The process from start to finish can take from 1-5 minutes but often, with the support of the device, reduction can occur in about 60 seconds. Patients who have previously had their shoulder dislocated often exclaim how much easier the relocation process was with the shoulder reduction device. They do not need to recuperate from sedation and can often leave the hospital after a brief rest. They are typically advised to visit an orthopedic clinic to be examined and receive X-rays to determine if there was any further damage from the dislocation. The shoulder reduction device saves time, reduces patient discomfort, can result in better outcomes, and moves patients through busy facilities more rapidly thereby serving more patients.

What is claimed is:

1. An adjustable shoulder support apparatus, comprising:
a removable lower rigid support base;
a removable flexible lower pad releasably coupled to a portion of the removable lower rigid support base;
a lower telescoping section coupled to the removable lower rigid support base;
an upper telescoping section residing at least partially within the lower telescoping section;
a locking mechanism configured to secure or lock the telescoping sections in a fixed height;
and an upper rigid support shaped in a fashion to substantially fit at least a portion of a human armpit.

2. The apparatus of claim 1, wherein the removable flexible lower pad comprises a flexible high friction pad.

3. The apparatus of claim 1, wherein an upper rigid support comprises a curved portion configured to mimic a curve or shape of an adult human armpit.

4. The apparatus of claim 1, wherein the height of the adjustable shoulder support apparatus is in a range from about 12 inches to about 36 inches or greater.

5. The apparatus of claim 1, wherein the upper rigid support further comprises a cushioned pad configured to cover at least a portion of the upper rigid support.

6. The apparatus of claim 1, wherein there are three or more telescoping sections arranged within each other.

7. The apparatus of claim 1, wherein there is a single support section that is not adjustable in height.

8. The apparatus of claim 1, wherein the telescoping section is secured by a detent button locking mechanism.

9. The apparatus of claim 1, wherein there are spikes on the lower rigid portion to engage with outdoor surfaces.

10. The apparatus of claim 1, wherein the telescoping sections comprise of a material selected from the group consisting of an alloy material, a metal material, an aluminum material, an injection molded plastic material, a composite reinforced material, a composite material, a composite reinforced plastic material, a wood material, a bamboo material, combinations of the same and the like.

11. The apparatus of claim 1, further comprising a shovel portion configured to be coupled to the lower telescoping section.

12. The apparatus of claim 1, wherein the upper and lower telescoping section comprises an adjustable length hiking or skiing pole.

13. The apparatus of claim 8, wherein the snow pole comprises one or more protrusions, spikes, and combinations of the same.

14. An adjustable shoulder support apparatus, comprising:
a removable lower rigid support base;
a removable flexible lower pad releasably coupled to a portion of the removable lower rigid support base;
a middle section comprised of a series of sections arranged as a scissor lift;
a locking mechanism configured to secure or lock the scissor lift to a set height;
and an upper rigid support shaped in a fashion to substantially fit at least a portion of a human armpit.

15. A method of using an adjustable shoulder support apparatus, comprising:
providing an adjustable shoulder support apparatus comprising a removable lower rigid support base; a removable flexible lower pad releasably coupled to a portion of the removable lower rigid support base; a lower telescoping section coupled to the removable lower rigid support base; an upper telescoping section residing at least partially within the lower telescoping section; a locking mechanism configured to secure or lock the lower telescoping section to the upper telescoping section; and an upper rigid support shaped in a fashion to substantially fit at least a portion of a human armpit;
adjusting the upper telescoping section such that the upper rigid support is nestled under an armpit of the user; and pulling down an arm of the user to reseat a shoulder.
